SEQUENCE LISTING (21 sequences)

SEQ ID NO: 1 - protein, 5 aa, Artificial sequence
  synthetic construct
IKVAV

SEQ ID NO: 2 - protein, 25 aa, Artificial sequence
  Synthetic construct
YESENGEPRGDNYRAYEDEYSYFKG

SEQ ID NO: 3 - protein, 6 aa, Artificial sequence
  Synthetic construct
GEPRDG

SEQ ID NO: 4 - protein, 10 aa, Artificial sequence
  Synthetic construct
ENGEPRGDNY

SEQ ID NO: 5 - protein, 16 aa, Artificial sequence
  Synthetic construct
YESENGEPRGDNYRAY

SEQ ID NO: 6 - protein, 4 aa, Artificial sequence
  Synthetic construct
KRSR

SEQ ID NO: 7 - protein, 4 aa, Artificial sequence
  Synthetic construct
RGDC

SEQ ID NO: 8 - protein, 4 aa, Artificial sequence
  Synthetic construct
RGES

SEQ ID NO: 9 - protein, 5 aa, Artificial sequence
  Synthetic construct
RGDFV

SEQ ID NO: 10 - protein, 5 aa, Artificial sequence
  Synthetic construct
RGDSP

SEQ ID NO: 11 - protein, 6 aa, Artificial sequence
  Synthetic construct
RGDSPC

SEQ ID NO: 12 - protein, 4 aa, Artificial sequence
  Synthetic construct
RGDS

SEQ ID NO: 13 - protein, 4 aa, Artificial sequence
  Synthetic construct
GRDC

SEQ ID NO: 14 - protein, 7 aa, Artificial sequence
  Synthetic construct
YRGDSPC

SEQ ID NO: 15 - protein, 5 aa, Artificial sequence
  Synthetic construct
GGRGD

SEQ ID NO: 16 - protein, 7 aa, Artificial sequence
  Synthetic construct
FHRRIKA

SEQ ID NO: 17 - protein, 10 aa, Artificial sequence
  Synthetic construct
RGDGGGKRSR

SEQ ID NO: 18 - protein, 20 aa, Artificial sequence
  Synthetic construct
KIPKASSVPTELSAISTLYL

SEQ ID NO: 19 - protein, 20 aa, Artificial sequence
  Synthetic construct
GWQDWIIAPEGYAAYYCEGE

SEQ ID NO: 20 - protein, 20 aa, Artificial sequence
  Synthetic construct
KPCCAPTQLNAISVLYFDDS

SEQ ID NO: 21 - protein, 20 aa, Artificial sequence
  Synthetic construct
AISVLYFDDSSNVILKKYRN

(19) United States
(12) Patent Application Publication
(10) Pub. No.: US 2010/0028387 A1
(43) Pub. Date: Feb. 4, 2010

(54) Biocompatible Coated Nanostructured Titanium Surfaces

(76) Inventors: Ganesan Balasundaram, Plymouth, MN (US); Tushar M. Shimpi, Plymouth, MN (US); Daniel M. Storey, Minneapolis, MN (US)

Correspondence Address: CHAMELEON SCIENTIFIC CORPORATION;AKA IONIC FUSION CORPORATION, 13355 10TH AVENUE NORTH, SUITE 108, PLYMOUTH, MN, 55441, US

(21) Appl. No.: 11/771,933

(22) Filed: Jun. 29, 2007

Related U.S. Application Data
(60) Provisional application No. 60/934,279, filed on Jun. 12, 2007.

(51) Int. Cl. A61K 9/00; A61K 38/02; A61K 38/07; A61K 38/08; A61K 38/16; A61K 38/14; C12N 5/00; A61K 35/12; A61P 43/00

(52) U.S. Cl. 424/400; 514/2; 514/18; 514/17; 514/12; 514/8; 435/402; 424/93.7; 977/700

(57) ABSTRACT

Bioactive molecules have been coated on nanotubular structured titanium substrates by molecular plasma deposition. The coatings promote cell adhesion and are particularly suited for orthopedic implants that provide improved bone cell adhesion and new tissue growth. Nanodimensional features on titanium substrates are engineered using electrochemical anodization techniques. The nanostructured surfaces provide superior support for a wide selection of polypeptide coatings.

This application claims benefit of U.S. Provisional Application Ser. No. 60/934,279 filed Jun. 12, 2007, which is hereby incorporated by reference herein in its entirety, including any figures, tables, nucleic acid sequences, amino acid sequences, or drawings.

### BACKGROUND OF THE INVENTION

1. Field of the Invention

The invention relates generally to biomaterials and particularly to bioactive protein-coated nanostructured titanium substrates.

2. Description of Background Art

Titanium and its alloys have been widely used to create dental and orthopedic implants because of their excellent biocompatibility and mechanical properties. Titanium (Ti) spontaneously forms an oxide layer up to a thickness of about 2 to 5 nm both in air and in the body, providing corrosion resistance. However, the normal oxide layer of titanium is not sufficiently bioactive to form a direct bond with juxtaposed bone, which may translate into a lack of osseointegration, leading to long-term failure of titanium implants.

In the past, many attempts have been made to improve the surface properties of Ti-based implants; e.g., by modifying Ti topography, chemistry, and surface energy, in order to better integrate into bone. Surface modification techniques include mechanical methods such as sand blasting, chemical methods such as acid etching, and the use of various coatings. A disadvantage of these approaches is that neither the mechanical nor the chemical methods produce highly controllable topological properties, and cell/tissue adherence may be unpredictable or insufficient for practical use. In some cases, the methods may cause formation of surface residuals, which can be interfere with osteoblast (bone forming cell) adherence and function. Bioactive materials covalently bonded to the surface have shown propensity for bone mineralization (Kirkwood, et al., 2003); however, the process involves functionalization of the substrate in order to covalently bond bioactive materials such as peptides.

One method of structuring titanium surfaces is the fabrication of titanium oxide (TiO₂) nanotube arrays by potentiostatic anodization of Ti foil (Paulose, et al., 2006). Nanotube lengths up to 134 μm have been achieved using fluoride ion solutions in combination with nonaqueous organic polar electrolytes, including dimethyl sulfoxide, formamide, ethylene glycol and N-methylformamide in the anodization process. Ti nanotube surface formation can be controlled to form specific diameters by optimizing fluoride concentrations during the electrolytic anodization process on TiO₂ (Bauer, et al., 2006).

Osteoblast adhesion is thought to be enhanced on substrate surfaces with smaller particle sizes and less surface crystallinity. Webster, et al., (2004) showed that osteoblast adhesion on nanophase titanium and titanium alloys is improved compared to growth on conventionally sized particles. The authors suggested that roughness increased particle boundaries at the surface, which caused adherence of an increased number of osteoblasts to the surface. More recently, Oh, et al. (2006) demonstrated improved adhesion/propagation of osteoblasts on vertically aligned laterally spaced nanoscale TiO₂ nanotubes.

Several workers have investigated bone mineralization on scaffoldings coated with polypeptides that contain sequences known to contain sequences that promote bone cell adhesion. Villard, et al. (2006), synthesized several multivalent RGD-containing molecules which, when immobilized on a substrate, promoted integrin αVβ3-dependent cell adhesion. One of the peptides reported was nearly 10-fold more efficient than fibronectin or vitronectin in promoting cell adhesion, and almost 100-fold more efficient than the linear RGD tripeptide. Grain size of hydroxyapatite coated Ti surfaces and RGD-functionalized hydroxyapatite strongly influenced anchorage cell adhesion (Balasundaram, et al. 2006).

Results reported by Kasai, et al (2004) also support the notion that RGD appears to be important for cell attachment. Those workers showed that an RGD sequence conjugated to IKVAV (SEQ ID NO:1) peptide formed a gel containing amyloid-like fibrils. The gel interacted with both integrins and IKVV (SEQ ID NO:7) receptor(s) and promoted cell adhesion. The researchers suggested that multifunctional amyloid-like peptide fibrils might serve as a basement membrane mimetic acting as a bioadhesive scaffold for tissue engineering. The work also indicated a role for RGD sequences as cell attachment foci for forming filopodia from amyloid-like fibrils.

Geistlich (2007) describes a multi-layer sheet of collagen membrane material with a smooth barrier face and an opposite fibrous face where collagen adheres to the fibrous face. The collagen layer contains cultivated bone forming cells such as osteocytes, osteoblasts, stromal or stem cells.

U.S. Pat. No. 6,291,428 discloses a method for promoting bone mineralization by administering polypeptides such as those containing the sequence YESENGEPRGDNYRAYEDEYSYFKG (SEQ ID NO:2) or polypeptides that contain the sequence GEPRDG (SEQ ID NO:3); ENGEPRGDNY (SEQ ID NO:4) or YESENGEPRGDNYRAY (SEQ ID NO:5). The peptide compositions were administered to the site where mineralization was desired; however, there was no evidence that these or other polypeptides immobilized on appropriate substrates could be used as effective scaffolds to promote bone mineralization.

Side chain peptides containing cell-binding sequences have been used to circumvent using surface attached native proteins that may undergo degradation and denaturation in vivo. Peptides covalently bound to glass, titanium and gold substrata enhance and accelerate the growth and differentiation of several different cell lines (Ferris, et al., 1999). Despite studies demonstrating that peptide-modified surfaces influence in vitro cellular behavior, it is recognized that in vivo use may raise issues of undesirable proteolysis and that manufacturing costs for the bound peptides may be high.

Methods to modify substrate surfaces for effective cell attachment and cell proliferation are needed. Well-defined surface properties suitable for attachment of bioactive molecules on implanted medical devices are especially desirable, both for promotion of tissue growth and for in situ placement of selected drugs.

Deficiencies in the Art

Despite progress in modifying metal surfaces to improve tissue and cell adhesion properties, adequate in vivo osseointegration on implant prostheses remains a challenge. Substrates that promote significant bone-tissue interactions with biomaterial surfaces over a period of time would be highly desirable. In order to ensure effective tissue adhesion, and thus clinical success of orthopaedic/dental implants, it is important to develop stable, biocompatible surfaces that enhance osteoblast functions for new bone formation. Additionally, the increasing importance of antimicrobial and other bioactive agents for in vivo implants requires improved materials and more effective means of releasing drugs at selected sites in the body.

### SUMMARY OF THE INVENTION

The present invention concerns peptide-coated nanostructured titanium and titanium alloy surfaces which exhibit high affinity for bone mineralization precursors. Using a molecular plasma deposition method, polypeptides comprising sequences that promote adhesion of anchorage-dependent cells and tissue building proteins have been deposited on the nanotubular structured surfaces. The polypeptides are firmly attached to the titanium surface tubules, which appear to be filled or partially filled with one or more peptides.

A particularly important aspect of the invention is the ability to coat and impregnate nanotubular engineered titanium surfaces with one or more selected bioactive molecules. This is accomplished using a molecular plasma deposition process, which is nondestructive of the bioactive material deposited onto the substrate surface. Importantly, the deposited polypeptides comprise sequences selected to enhance attachment and proliferation of osteoblast cells.

Nanotubular titanium metal surfaces can be produced using known anodization procedures. This process provides modified titanium surfaces covered with nanotubular structures with defined open ends and lengths, which can be changed by modifying anodization conditions. The surface characteristics of the titanium can be controlled to create nanotubular surfaces with predictable characteristics. Nanotubular titanium and titanium alloy surfaces are well suited for coating with biomaterials for orthopedic applications.

The present work shows that the open ends of the nanotubes can be filled or partially filled with biomaterials, for example peptides, which adhere to the inner and/or outer surfaces of the nanotubes to a greater or lesser extent depending on the dimensions of the nanotubes and the deposited biomaterial. Properly selected bioactive substances will not only be deposited on the nanotube surface, but also “fill” the tubes allowing increased amounts of a material to be attached to the surface. When deposited to the substrate surface using the described molecular plasma deposition, the attachment of the biomaterial is non-covalent. This provides a route to developing time-release of some materials and is expected to be applicable to a wide range of small molecules such as drugs.

Accordingly, whether immobilized inside or outside the nanotubes, biomaterials can be released in a time-dependent fashion in vivo. This is particularly attractive for use in various implants and becomes possible because the biomaterials are not covalently attached to the substrate surface. Nanotube features can be designed so that adherence of the coating can be modified to be relatively weak or, by adjusting the size of the tubes, change the ratio of bioactive material inside and outside the tubes.

While the invention has been illustrated with a nanotubular titanium surface, the nanotube features can be created on titanium-based substrates; e.g. nickel/titanium, and various titanium compositions with molybdenum, zirconium, niobium, aluminum, iron, vanadium, and tantalum. Several of these alloys are currently used to fabricate implant devices; for example, Ti29Nb13Ta4.6Zr. Non-titanium containing materials are also contemplated; e.g., Nitinol.

When used as coatings on implants, the biomolecules act not only as anchors for osteoblasts but also enhance bone growth in vivo. As demonstrated herein, the RGD tripeptide sequence was deposited on a nanostructured Ti surface and showed strong cell adhesion for osteoblasts and fibroblasts.

### BRIEF DESCRIPTION OF THE FIGURES

FIG. 1 is a schematic of an anodization apparatus; two electrode configurations are linked to DC power supply. A platinum disk and Ti disks serves as cathode and anode respectively. 1.5% HF was used as electrolyte contained in a Teflon beaker.

FIG. 2A shows formation of nano-tubular features on Ti substrates at low magnification. Bars=5 μm.

FIG. 2B shows formation of nano-tubular features on Ti substrates at high magnification. Bars=500 nm.

FIG. 3 is a representative sketch of the MPD chamber used to coat RGDS and RGES peptides on anodized Ti substrates. FIG. 3A shows the molecular plasma deposition apparatus: vacuum chamber 1; high voltage power supply 2, substrate holder 3; substrate 4; high voltage power supply 5; needle 6; feeder tube to needle 7; orifice 15 into reservoir 8; colloidal liquid suspension 9. FIG. 3B is a modification of the apparatus of FIG. 3A: vacuum chamber 1; high voltage power supply 2; substrate holder 3; substrate 4; high voltage power supply 5; needle 6; feeder tube 7; orifice 15 into reservoir 8; liquid suspension 9; secondary chamber 10; secondary chamber gas supply 11; secondary chamber gas supply line 12; pressure regulator 13; gas line from regulator 14.

FIG. 4 is an XPS spectrum of (a) unanodized, (b) anodized, and peptide coated (c) substrates.

FIG. 5A shows fluorescent images of increased osteoblast adhesion on anodized Ti coated with cell adhesive peptide RGDS (SEQ ID NO: 13), Stain=DAPI. Bars=200 μm.

FIG. 5B shows of increased osteoblast adhesion on anodized Ti coated with non cell adhesive peptide RGES (SEQ ID NO: 9). Stain=DAPI. Bars=200 μm.

FIG. 6 shows increased osteoblast adhesion on anodized Ti coated with cell adhesive peptide RGDS (SEQ ID NO: 13). Values are mean±SEM; n=3; *p<0.01 compared to anodized Ti; **p<0.01 compared to non cell adhesive peptide RGES (SEQ ID NO: 9) coated Ti.

FIG. 7A is a surface analysis of peptide coated Ti compacts using CBQCA analysis of uncoated Ti. Magnification is 10×.

FIG. 7B is a surface analysis of peptide coated Ti compacts using CBQCA analysis of RGDS-coated Ti. Magnification is 10×.

FIG. 7C is a surface analysis of peptide coated Ti compacts using CBQCA analysis of KRSR-coated Ti. Magnification is 10×.

FIG. 7D is a surface analysis of peptide coated Ti compacts using CBQCA analysis of IKVAV-coated Ti. Magnification is 10×.

### DETAILED DESCRIPTION OF THE INVENTION

Bioactive agents on nanotubular structured titanium (Ti) surfaces are disclosed. A model cell-adhesive peptide, RGDS (SEQ ID NO: 13), was attached to a Ti (nanotubular) surface by a molecular plasma deposition (MPD) process. The MPD-peptide coatings on Ti were characterized by X-ray photoelectron spectroscopy (XPS) which showed that the coatings were attached to the surface of the substrate. Results showed greater osteoblast adhesion on the nanotubular titanium coated with a cell binding peptide such as RGDS (SEQ ID NO: 13) compared to unanodized Ti or flat Ti surfaces coated. Binding of cells to any of the Ti surfaces coated with the negative control peptide RGES (SEQ ID NO: 9) was significantly less.

The present invention provides peptide coated nanotubular structured titanium surfaces ideally suited for implants where surfaces promoting osteoblast cell attachment and proliferation are needed. The disclosed biosurfaces are suitable for use on implants such as orthopedic devices and dental implants.

Cell Adhesion to Identified Amino Acid Sequences

Naturally occurring in vivo mineralization components, including polypeptides, have been investigated by others as suitable scaffolds for bone cell attachment. One approach to increasing osteoblast adhesion to surfaces has been to covalently bind selected peptides on traditional micron-structured or flat materials in attempts to develop a more osteogenic inducing surface. Rezania and Healy (1999) reported some success in reducing interfacial fibrous tissue and improving osseous differentiation on a metallic surface.

Protein-mediated adhesion mechanisms involve interactions of select cell-membrane receptors with specific protein domains (or peptide sequences) such as arginine-glycine-aspartic acid (RGD) and lysine-arginine-serine-arginine (KRSR). These peptides either exist in nature as in the case of RGD, or like KRSR (SEQ ID NO:6) have been designed to elicit responses from specific cell lines; for example, from osteoblasts, but not from fibroblasts. Fibroblasts are responsible for fibrous tissue formation which is undesirable for most implants.

Extracellular matrix proteins that contain the cell binding domain RGD have a major role in cell behavior because they regulate gene expression by signal transduction set in motion by cell adhesion to the biomaterial. Collagen, fibronectin and vitronectin proteins are particularly important in mediating osteoblast adhesion; moreover, RGD is part of the structure in all three of these proteins and is recognized by cell membrane integrin receptors.

The work of several researchers, e.g., Kasai, et al (2004), supports the notion that RGD is important for cell attachment. The researchers demonstrated that an RGD sequence conjugated to IKVAV (SEQ ID NO:1) peptide formed a gel containing amyloid-like fibrils. The gel interacted with both integrins and IKVV (SEQ ID NO:7) receptor(s) and promoted cell adhesion. The authors suggested use of multifunctional amyloid-like peptide fibrils to serve as a basement membrane mimetic acting as a bioadhesive scaffold for tissue engineering.

Based on the known cell binding properties of RGD, several α-helical peptides are expected to be useful as surface-immobilized agonists to promote cell adhesion, particularly those that form fibril-like structures carrying an amino terminal RGD motif. Other active peptides which recognize different receptors including integrins and proteoglycans, may also form multifunctional amyloid-like fibrils; e.g., KRSR.

Several RGD peptides are known and have been tested for recognition by integrin. The RGD peptides include RGDSP (SEQ ID NO:11), RGDSPC (SEQ ID NO:12), RGDS (SEQ ID NO:13), GRDC (SEQ ID NO:14), YRGDSPC (SEQ ID NO:15), (G)nRGD (SEQ ID NO:16) and cyclo(-RGDfV) (SEQ ID NO:10). The cyclic RGD pentapeptides in which D-amino acids follow the aspartic acid residue, have a conformation that is best recognized by integrins and by osteoblast and osteoprogenitor cells. These peptides have a reduced affinity to the platelet receptor, aIIbb3. A hydrophobic residue in this position; e.g. phenylalanine, contributes to activity and selectivity.

KRSR (SEQ ID NO:6) is a heparin sulfate (HS) binding protein. HS is a linear polysaccharide found in all animal tissues. It occurs as a proteoglycan in which two or three HS chains are attached in close proximity to the cell surface or extracellular matrix protein. FHRRIKA (SEQ ID NO:17) is also a HS binding protein. It is thought that utilizing peptide sequences incorporating both cell and heparin-adhesive motifs can enhance cell surface interactions and influence the long term formation of mineralized ECM in vitro.

Chemical mediators of bone growth (such as growth factors) are essential in the body. Growth factors (GFs) are peptides that regulate cell growth, function and motility, resulting in the formation of new tissue. Bone GFs influence the synthesis of new bone by acting on the local cell population present in bone marrow and on bone surfaces. They act directly on specific osteoblasts by regulating local cell function, inducing angiogenesis (examples include: basic fibroblast growth factors 1 and 2, bFGF-1/2, vascular endothelial growth factor, VEGF, etc.), and by promoting osteogenesis by increasing endothelial and osteoprogenitor cell migration and differentiation (Urist, 1965). Bone matrix contains a number of growth factors, including fibroblast growth factors (FGFs), insulin-like growth factor I and II (IGF-I, IGF-II), platelet-derived growth factors (PDGF), and the transforming growth factor beta (TGF-β) supergene family (which currently has 43 members and includes, among others, TGF-β1-5 and the bone morphogenic proteins, BMP 2-16) (Burt et al., 1994).

The proteins of the TGF-β superfamily regulate many different biological processes, including cell growth, differentiation and embryonic pattern formation (Zhu et al., 1999). BMPs play a critical role in modulating mesenchymal cell differentiation by inducing the complete sequence of endochondral bone formation where cartilage forms first and is subsequently replaced by bone (Wozney et al., 1998). Other GFs (such as TGF-β, IGF and FGFs) all affect fully differentiated bone-forming cells, causing them to divide or increase secretion of extracellular matrix and proteins. In contrast, BMPs are the only known GFs with the ability to stimulate the differentiation of mesenchymal stem cells into the chondroblastic and osteoblastic direction (Chen et al., 1991). Therefore, because of the presence of GFs, the proximity, chemical composition, and quality of the local bone are less of a factor in bone regeneration.

Recombinant BMPs have a broad therapeutic potential for orthopedic reconstruction. However, different BMPs are not identical in their osteoinductive potential. For example, BMP 5 is needed in larger amounts to induce the same amount of bone compared with BMP 2 or 7 and only BMP 7 (otherwise known as OP-1) has been shown to regulate Cbfal, which has been identified as the only transcriptional factor responsible for osteoblastic differentiation and expression of osteocalcin and osteopontin, which are proteins important for osteoblast differentiation (Chen et al., 1991).

Native BMP is present in cortical bone in minute amounts (specifically, 1-2 μg of BMP per kg of cortical bone). While recombinant human (rh) BMP 2, 4 and 7 have been shown to induce bone formation in many experiments and are now also being tested in clinical studies (Boden, 1999), the amount of rhBMP 2 necessary to produce bone induction in vivo is on the order of 0.7-17 μg of BMP per mg of a collagen carrier while the activity of rhBMP 2 is one-tenth that of purified human BMP 2 (Bessho, et al., 1999). This suggests that native BMP activity is either a combination of the activities of different BMPs or that it is the synergistic activity between them (Wozney et al., 1990). This leads to some concerns regarding the use of BMPs at such high concentrations (Poynton et al., 2002).

Use of Titanium-Based Immobilization Substrates

While titanium and its alloys are widely used in orthopedic and dental applications, the titanium oxide surface that forms when the metal is exposed to air is not sufficiently bioactive to bond with bone. There is little osteoblast adhesion to smooth or microtextured titanium; however, Webster and Ejiofor (2004) have demonstrated increased osteoblast adhesion on nanophase titanium metals. Recently, Oh, et al. (2006) demonstrated accelerated osteoblast cell growth on TiO₂ nanotubes.

Nanotube surface characteristics of Ti can be controlled by modification of the anodization process. Nanotube diameter can be controlled by electrolytic solution composition, time of anodization, and temperature at which the anodization is conducted. Larger diameter nanotubes, for example, may be used to accommodate large biomolecules or multiply deposited materials. Pore diameters ranging from 20 to 500 nm with varying wall thicknesses are readily synthesized, making it possible to load larger molecules into the nanotubes.

Nanotube length (height) can also be controlled so that the titanium nanotube surface is relatively uniform. Uniformity provides a more level surface on which depth of deposited biomolecule layers can be better controlled. Unanodized titanium surfaces lacking nanotubular structure show little tendency to attract osteoblast cells.

The nanotubular titanium surface produced under the described anodization conditions is more compatible with natural bone than the micropatterned surfaces commonly found on orthopedic implants. As discussed, both length and nanotube diameter can be changed to accommodate desired deposited materials, such as the different types of collagen, and other protein based compounds, whether natural or synthetic, that may be suitable for enhancing osteoblast adhesion and bone growth. Some modifications in the diameter and length of the nanotubes formed on Ti surfaces by etching processes can be made so that pore diameter can range from about 30 to over 500 nm (Grimes, 2006). By using selected anodization conditions, titanium surfaces that mimic features of natural bone can be provided. Type I collagen is the main organic component of bone, exhibiting a triple helix 300 nm in length, 0.5 nm in width and a periodicity of 67 nm. All type I collagen dimensions and inorganic bone components are compatible with the dimensional aspects of the nanostructured titanium surface

The present invention shows that nanotubular titanium surfaces on which selected peptides have been deposited have stable surfaces that enhance cell adhesion compared to cell adhesion on uncoated nanotubular titanium surfaces. Cell adhesion is increased compared to conventional titanium surfaces, as one might expect, but adhesion and proliferation of osteoblast cells are even further increased compared to adhesion on uncoated nanotubular titanium surfaces. Apparently the polypeptides are strongly adhered to the nanostructured surface and in turn attract and enhance cell attachment to the RDG and other cell attachment domains. It is believed that the nanotubes themselves may partially or completely fill with the cells, thereby providing a wide area for cell attachment and proliferation.

Coating titanium nanotubular substrates with peptides containing cell binding motifs were investigated as candidates for enhancing osteoblast adhesion and potential bone regeneration.

Several cell binding domains incorporated into a variety of peptides were considered as appropriate surfaces for cell adhesion. KRSR sequences selectively bind transmembrane proteoglycans of osteoblasts and are expected to be useful in orthopedic applications. Other peptides include RGDSP (SEQ ID NO:11), RGDSPC (SEQ ID NO:12), RGDS (SEQ ID NO: 13), RGDC (SEQ ID NO:8), YRGDSPC (SEQ ID NO:15), (G)nRGD (SEQ ID NO:16), and cyclo(RGDfV) (SEQ ID NO: 10) where f represents the d-enantiomer of phenylalanine. Cyclic RGD pentapeptides in which d-amino acids are located adjacent to an aspartic acid residue have a confirmation that is best recognized by integrins and have a much reduced affinity to the platelet receptor, aIIbb3. In addition to d-amino acids, hydrophobic residues in this position; for example, phenylalanine, increase activity and selectivity.

Heparin binding motifs are also known to bind cells. KRSR, for example, is a linear polysaccharide found in all animal tissues. It occurs as a proteoglycan in which two or three HS chains are attached in close proximity to cell surface or extracellular matrix proteins. FHRRIKA (SEQ ID NO:16) is another example of a heparin binding protein (HBP). Peptide sequences incorporating both cell- and heparin-adhesive motifs are expected to be useful in enhancing the degree of cell surface interactions and to influence long-term formation of mineralized ECM.

There are several other biomolecules that are likely to be useful in improving tissue biomaterial interactions. Bone sialoprotein (BSP) is found in mineralized tissues including bone, dentin, cementum and hypertropic cartilage. BSP contains a C-terminal cell attachment sequence (RGD) and two glutamic acid domains. BSP is known to nucleate hydroxyapatite formation and to stimulate bone remodeling, as well as to mediate attachment of fibroblasts, osteoblasts and osteoclasts.

Bone morphogenetic proteins (BMPs) play an important role in bone formation, particularly BMP-2, BMP-3, BMP-4, BMP-7, BMP-12, BMP-13 and BMP-14.

Bone morphogenetic protein-2 (BMP-2) has a broad therapeutic potential for orthopedic reconstruction. BMP-2 can induce bone formation and regeneration during early embryonic development. BMP-2 is synthesized as a large precursor molecule first and the mature protein is secreted by proteolytic cleavage. The wrist epitope of the BMP-2 is thought to bind BMP receptor 1A based on crystal structure studies. It has been suggested that the knuckle epitope binds the BMP receptor type II.

Bone morphogenetic protein-7 (BMP-7) or OP-1 regulates Cbfa, which has been identified as a transcriptional factor responsible for osteoblastic differentiation and expression of osteocalcin and osteopontin, proteins important for osteoblast differentiation. Other transcriptional factors may also be important in regulating genes responsible for bone formation/regulation, including transcriptional factor Runx2.

Improved Cell-Adhesion Coatings

After identifying numerous peptides to test for osteoblast adhesion and proliferation, the difficulty of coating several cell-adhesion peptides on a substrate surface was addressed. In general, there are few effective methods other than covalent attachment to effectively attach peptides to a metal surface while maintaining the functional properties of the peptide. Additionally, an attached amino acid sequence should have sufficient surface exposure to effectively bind a bone-forming cell, such as an osteoblast, or in vivo, a bone-forming protein precursor.

It was found that biomolecules involved in adhesion of bone-forming elements can be deposited on substrate surfaces using a modified molecular plasma deposition (IPD) method. The selected peptide or protein was solubilized in a liquid, either as a solution or colloidal suspension, and deposited from an ionic plasma generated as a corona from a high voltage needle tip onto a substrate. The deposited peptide or protein maintains structural integrity after deposition and acts as a stable scaffold to which bone-forming elements will readily attach.

To ensure high adherence, the peptides were deposited on a nanostructured surface. Nanotubular Ti surfaces were found to provide ideal surfaces because they have reproducible and controllable properties and have the physical surface characteristics that allow strong cell adhesion. XPS confirmed that the peptides were firmly deposited on the Ti surface.

Using the molecular plasma technique as described in Example 2, several polypeptides were coated onto Ti nanotubular substrates. RGDS and RGES were synthesized with the expectation that surface-attached RGDS would readily attract osteoblast cells while RGES, where E replaces D, showed no effect in attracting the cells.

Cell Adhesion

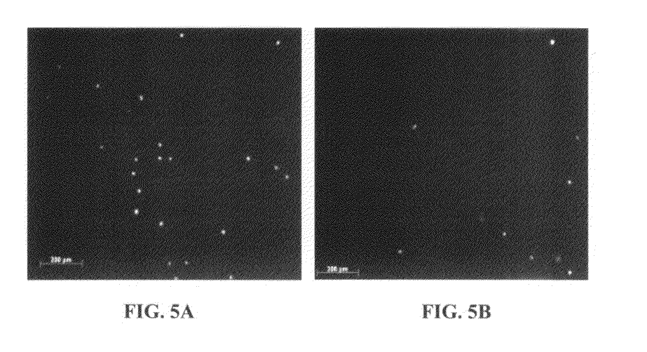

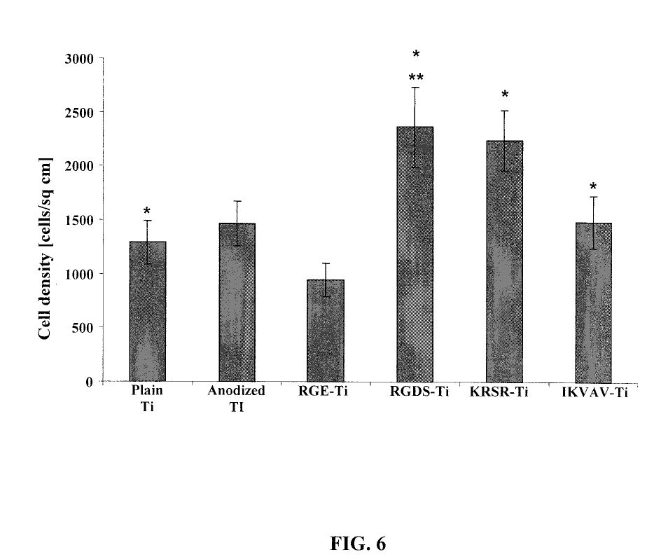

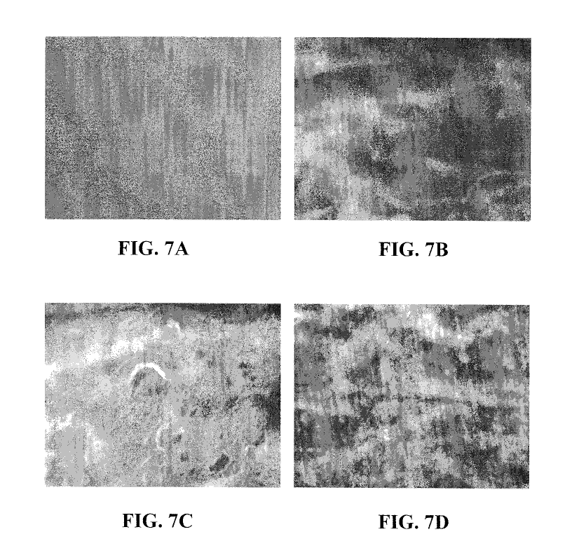

Following a 4 hour incubation, osteoblasts showed greater adhesion to anodized Ti coated with the RGDS using MPD process compared to RGE (negative control peptide) coated Ti (FIG. 5 and FIG. 6) or unanodized Ti samples. Results provided evidence of increased osteoblast adhesion on anodized Ti compared to unanodized Ti (FIG. 6). Cell attachment activity of osteoblasts appeared to be significantly better with Ti coated with bioactive molecules using the MPD process.

Polypeptides KRSR (SEQ ID NO: 6) and IKVAV (SEQ ID NO:1) were deposited on Ti nanotubular substrates and tested similarly to RGDS (SEQ ID NO: 13).

Peptide Coating and Characterization

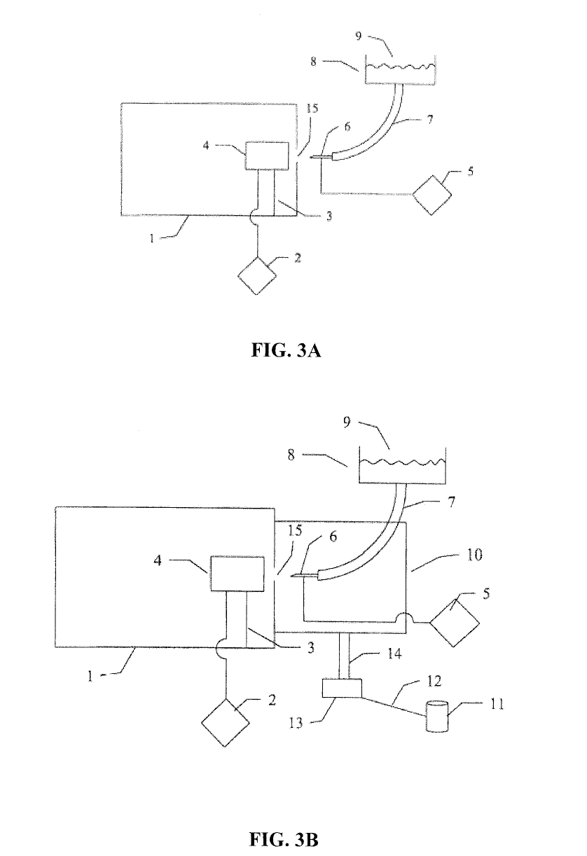

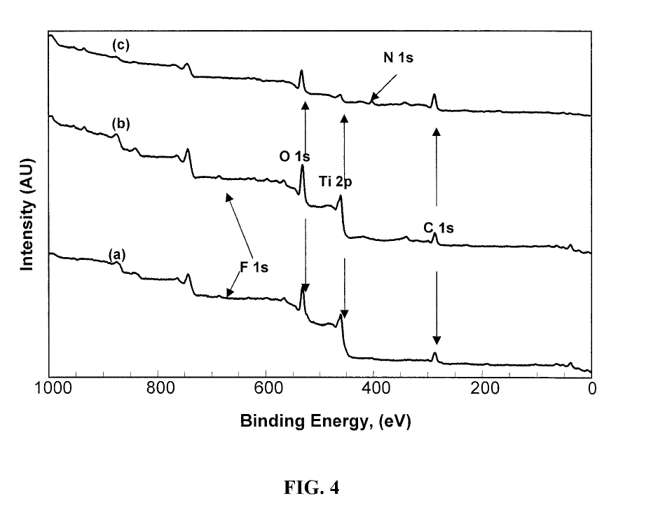

FIG. 3 shows the schematic diagram of a MPD device used for peptide coatings according to embodiment of the present work. XPS was taken on each Ti sample to examine Ti 2p binding energy (Table 1). Importantly, for anodized samples, other than TiO₂, no other titanium species were present. XPS also provided the evidence that the layers of oxide mainly contained C, O, Ti, and F (Table 1) and were similar between the unanodized and nanotubular anodized Ti.

TABLE 1
Atomic percentage of select elements of unanodized,
anodized, and peptide coated Ti substrates as examined
by X-ray Photoelectron Spectroscopy.

| Substrate | C | O | Ti | N | F |
|---|---|---|---|---|---|
| Unanodized Ti | 40.2 | 41.1 | 17.5 | — | 1.2 |
| Anodized Ti | 35.6 | 45.0 | 16.6 | — | 2.0 |
| RGDS Coated Ti | 59.8 | 26.6 | 3.2 | 6.0 | — |

XPS analysis was performed on uncoated and coated peptide coated surfaces to ensure the presence of RGDS on the Ti surfaces, and the results were compared to that of unmodified surfaces. No nitrogen was detected on the uncoated surfaces, and carbon was present due to impurities from processing. There was a distinct increase in C1s (287 eV) and emergence of N1s (402 eV) peaks with modification of RGDS. This was also followed by consequent decrease in Ti 2p (460 eV) peaks for the above surfaces due to the surface coverage by the peptide molecules. For specific cell recognition, this degree of coverage has a significant effect on cell-surface adhesion.

### EXAMPLES

The following examples are provided as illustrations of the invention and are in no way to be considered limiting.

Materials and Methods

Bone Growth Factors

Peptides: RGDC, Cyclo(RGDfV), RGDSPC, KRSR, FHRRIKA, RGDGGGKRSR (SEQ ID NO:18), KIPKASSVPTELSAISTLYL (SEQ ID NO:19) (from BMP-2 knuckle epitope), GWQDWIIAPEGYAAYYCEGE (SEQ ID NO:20) (from BMP-7) KPCCAPTQLNAISVLYFDDS (SEQ ID NO:21) (from BMP-7), AISVLYFDDS SNVILKKYRN (SEQ ID NO:22) (from BMP-7)

Proteins: rhBMP-2 protein, rhBMP-7 protein or OP-1, Vitronectin

Cell adhesive peptides: RGDS (arg-gly-asp-ser) SEQ ID NO. 13; KRSR (lys-arg-ser-arg) SEQ ID NO. 6; IKVAV (Ile-lys-val-ala-val) SEQ ID NO. 1.

Cell Cultures

Cell cultures Fibroblasts (CRL-2317, American Type Culture Collection), osteoblasts (CRL-11372, American Type Culture Collection), and Endothelial Cells (VEC Technologies, Rensselaer, N.Y.) were used in the cell adhesion tests.

In Vitro Cell Adhesion Procedure

Substrates were rinsed with phosphate buffered saline (PBS) (1× strength) before seeding the cells. The cells were cultured on the substrates in Dulbecco's Modified Eagle Medium (Hyclone) supplemented with 10% fetal bovine serum (Hyclone) and 1% penicillin/streptomycin (Hyclone) with an initial seeding density of 3500 cells/cm² of substrate. Cells were then allowed to adhere on the substrates under standard cell culture conditions (37° C. temperature, 5% CO₂ and 95% humidified air) for 4 hours.

After the prescribed time period, the cell culture medium was aspirated from the wells and the substrates gently rinsed with PBS three times to remove any non-adherent cells. The adherent cells were then fixed with a 4% formaldehyde solution (Fisher) and stained with a Hoescht 33258 dye (Sigma). The cell numbers were counted under a fluorescence microscope.

X-ray Photoelectron Spectroscopy (XPS)

Peptide coatings were characterized by XPS. Spectra were recorded with a PHI 555 spectrometer on plain and peptide coated compacts using a monochromatized Al Kα X-ray and a low energy electron flood gun for charge neutralization. Survey spectra were collected from 0 to 1000 eV with pass energy of 160 eV and a take-off angle of 55°. Graphics Viewer program was used to determine peak areas. The compacts were mounted on a sample stub with conductive carbon tape. After acquisition, fitting was then completed with software provided by PHI 555; binding energy (BE) values were ±0.2 eV.

Fluorescence with 3-(4-carboxybenzoyl)quinoline 2-carboxaldehyde (CBQCA) Fluorescence

Surface peptide coatings were characterized by CBQCA (Molecular Probes, USA) fluorescence technique. CBQCA reagent solutions were prepared by dissolving the reagent in methanol (3 mg/ml). Potassium cyanide (Aldrich Chemical Inc, USA) was dissolved in water to give a 10 mM solution. Substrates were exposed to CBQCA and potassium cyanide stock solutions for 2 h at room temperature. Inherently CBQCA is nonfluorescence molecule but upon reaction with amine groups in the presence of cyanide molecules, it fluoresces well. Coated substrates were visualized under a fluorescence microscope (Leica, DM IL) with 10× magnification to ascertain peptide coatings. Images were obtained using QCapture software.

### Example 1

### Anodized Titanium

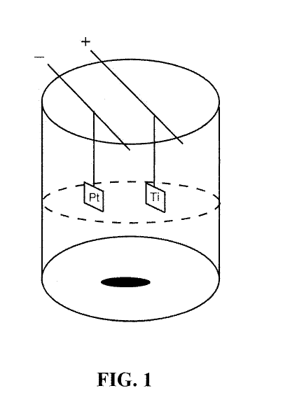

A standard anodization apparatus utilizing a platinum cathode and titanium anode connected by copper rods to a power supply was employed (FIG. 1). The beaker is Teflon® or other material impervious to the acid.

Rectangular shaped titanium foil with a thickness of 250 μm (99.7%; Alfa Aesar) was cleaned ultrasonically with ethanol and water prior to anodization. Cleaned substrates were etched with a mixture of 1M HNO₃ (Aldrich) (with few drops of HF solution) and further cleaned with deionized water. Afterwards, pretreated specimens were anodized in a 1.5% hydrofluoric acid (HF) solution. Using a DC power supply, a 20 V anodizing voltage was applied for 10 minutes. During processing, the anode and cathode were kept parallel with a separation distance of about 1 cm. Specimens were rinsed with deionized water and dried with nitrogen gas immediately after being anodized. Before osteoblast adhesion was performed, specimens were sterilized under UV for 1 hr in a laminar flow hood.

Alternatively, etching time may be carried out for minutes to hours and/or the electrolyte can be hydrofluoric acid (HF) or mixtures of HF with dimethylsulfoxide (DMSO) in various ratios. Such modifications result in nanotube structures having different tube diameters and heights.

The surfaces of the substrates were characterized by scanning electron microscopy (SEM). For SEM, substrates were first sputter-coated with a thin layer of gold using an Ernest Fullam Sputter Coater (Model; AMS-76M) in a 100 mTorr vacuum argon environment for a 3 min period and 10 mA of current. Images were taken using a TESCAN-MIRA/LSM SEM at a 20 kV accelerating voltage. Digital images were recorded using the TESCAN-MIRA software.

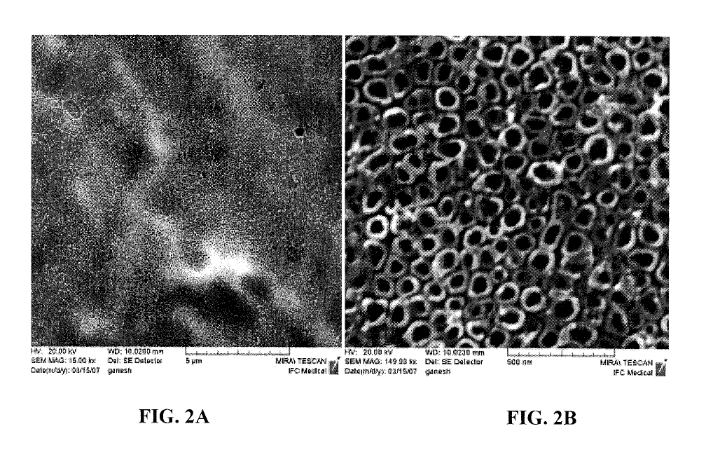

FIG. 1 shows the schematics of potentiostatic anodization used to produce nanotubular structures on Ti samples. After anodization in 1.5% HF at 20 V for 10 min. the Ti surface was oxidized and possessed nanotubular features uniformly distributed over the whole surface (FIG. 2A). High magnification SEM images showed that the inner diameter of the nanotubular structures was about ˜70 nm with a wall thickness of ˜15 nm (FIG. 2B).

X-ray Photoelectron Spectroscopy (XPS)

Peptide coating was confirmed by XPS. Spectra were recorded with a PHI 555 spectrometer on plain and peptide coated compacts using a monochromatized Al Kα X-ray and a low energy electron flood gun for charge neutralization. Survey spectra were collected from 0 to 1000 eV with pass energy of 160 eV and a take-off angle of 55°. Graphics Viewer program was used to determine peak areas. The compacts were mounted on a sample stub with conductive carbon tape. After acquisition, fitting was then completed with software provided by PHI 555; binding energy (BE) values were ±0.2 eV.

Bioactive Molecule Coating Distribution

In order to determine the distribution of coated biomolecule groups on anodized Ti substrates, fluorescence methods, and X-ray photo electron spectroscopy (XPS) were used.

For the fluorescence method, randomly selected functionalized substrates are stained using a CBQCA amine-labeling kit (Molecular Probes, Eugene, Oreg.) following manufacturer instructions and then visualized by fluorescence microscopy. CBQCA is a non-fluorescence molecule but upon reaction with amine groups in the presence of cyanide molecules, exhibits fluorescence. Images can be obtained using software interfaced with fluorescence microscopy. Alternatively, in order to detect the peptide coatings to the anodized Ti surface, the peptide RGD is labeled by fluorescein isothiocyanate (FITC) to the amino terminus of the peptide as the fluorescent probe.

FIG. 2A is a scanning electron microscope image of an unmodified titanium surface. FIG. 2B shows a titanium surface after the anodization treatment. The diameter of the nanotubes on the anodized titanium is approximately 70 nm and length approximately 200 nm.

Surface roughness of anodized titanium is about 25 nm, compared with unanodized titanium, which is on the order of 5 nm. Roughness is determined by Ra values measured by SEM analysis of gold sputtered anodized substrates. Selected kV can be used to obtain images of substrate topography at low and high magnification so that pore geometry and surface feature size can be observed. Surface roughness can be quantified using atomic force microscope interfaced with imaging software. A scan rate, for example 2 Hz, can be used at a selected scanning point; e.g., 512, to obtain root mean square roughness values. Scans can be performed in ambient air at 15-20% humidity.

The titanium surface can be further characterized using X-ray photoelectron spectroscopy (XPS) to determine peptide film thickness, density and coverage. While useful for peptide characterization, coatings prepared from larger protein molecules are preferably characterized with fluorescence techniques; for example, CBQCA (3,4-carboxybenzoyl quinoline-2-carboxaldehyde) and BCA (bicinchoninic acid) assays.

### Example 2

### Molecular Plasma Deposition Apparatus

The deposition apparatus includes a vacuum chamber with a small aperture, and a small bore, metallic needle connected to a tube connected to a reservoir holding a liquid suspension or solution of the material desired to be deposited. The reservoir is at atmospheric pressure. A power supply with the ability to supply up to 60 kV can be employed; however, the voltage attached to the needle is typically −5000 volts to +5000 volts. A substrate inside the vacuum chamber, is centered on the aperture with a bias from −60 kV through −60 kV, including ground. The apparatus is illustrated in FIG. 3A.

Another molecular deposition apparatus is illustrated in FIG. 3B. This is a modification of the apparatus in FIG. 3A such that the needle, tube, and reservoir are disposed in an enclosure that excludes air, but allows for the controlled introduction of other gases. Optionally selected gases include argon, oxygen, nitrogen, xenon, hydrogen, krypton, radon, chlorine, helium, ammonia, fluorine and combinations of these gases. The system can be operated at a pre-determined pressure above or below atmospheric pressure. While atmospheric pressure is generally preferred for generation of the plasma, reduced pressure up to about 100 mTorr may in some instances provide satisfactory depositions.

In the apparatus shown in FIG. 3, the pressure differential between the corona discharge and the substrate is about one atmosphere. The outside pressure of the vacuum chamber is approximately 760 Torr, whereas pressure in the area of the substrate is approximately 0.1 Torr.

### Example 3

### Osteoblast Cell Adhesion to Anodized Titanium

Human osteoblasts (CRL-11372 American Type Culture Collection, population numbers 7-8) in Dulbecco Modified Eagle Medium (Gibco) supplemented with 10% fetal bovine serum (Hyclone) and 1% Penicillin/Streptomycin (Hyclone) were seeded at a density of 3500 cells/cm² onto an anodized titanium substrate and placed in standard cell culture conditions (humidified, 5% CO₂/95% air environment) for 4 hr. The substrate was rinsed in phosphate buffered saline to remove any nonadherent cells. The remaining cells were then fixed with formaldehyde (Aldrich Chemical Company, USA), stained with Hoescht 33258 dye (Sigma), and counted under a fluorescence microscope (Leica, DM IRB). Five random fields were counted per sample substrate. All tests were run in triplicate and repeated at least three separate times. Standard t-tests were used to check statistical significance between means.

Results showed significantly increased (p<0.01) osteoblast adhesion on anodized Ti compared to unanodized Ti after 4 hr exposure to the cells, FIG. 6.

### Example 4

### Osteoblast Adhesion to Peptide Coated Nanotubular Titanium

HPLC purified RGDS peptide was obtained from American Peptide Company (Sunnyvale, Calif.). 1 mmole stock solution was prepared in deionized water. Anodized Ti substrates were placed inside a molecular plasma deposition mounting chamber (FIG. 3). To minimize the variables in the experiments, the power supply was kept constant and the voltage measured by the probe between needle and the chamber (ground) was 5 KV. The chamber was evacuated to a pressure of X Torr. After the peptide was deposited onto the substrate, the chamber was vented to the atmosphere. The peptide coated titanium was removed and vacuum dried for a day then stored in a desiccator until further use.

KRSR and IKVAV were deposited under the same conditions as RGDS. A non-cell binding peptide, RGE, was obtained from American Peptide Company and coated by the same method onto anodized nanostructured titanium.

Osteoblasts cells (3,500 cells/cm2) in DMEM (in the presence of 10% fetal bovine serum) were seeded per substrate and allowed to adhere in a 37° C., humidified, 5% CO₂/95% air environment for 4 hours. At the end of the prescribed time period, non-adherent cells will be removed by rinsing in phosphate buffered saline. Adherent cells on the substrates were fixed with 4% formaldehyde in sodium phosphate buffer; the cell nuclei was stained with DAPI, visualized and counted using fluorescence (365 nm excitation; 400 nm emission) microscopy with image analysis software.

Cell density (cells/cm²) was determined by averaging the number of adherent cells in five random fields per substrate. Each cell adhesion experiment was run in triplicate and repeated at three separate times.

FIG. 6 compares results of osteoblast adhesion on nanostructured titanium surfaces coated with RGE, RGDS, KRSR or IKVAV. Smooth surface unanodized titanium, shows a cell density of about 1200 cells/cm² while anodized uncoated titanium has a cell density of about 1500 cells/cm². RGE-coated anodized titanium has less than 900 cells/cm², while both RGDS and KRSR coated titanium showed significantly greater osteoblast adhesion compared to anodized nanostructured titanium.

All experiments were run in triplicate and were repeated three different times. Numerical data were analyzed using t-test; statistical significance was considered at p<0.05.

Sequence Listing

| | |
|---|---|
| SEQ ID NO: 1: | IKVAV |
| SEQ ID NO: 2 | YESENGEPRGDNYRAYEDEYSYFKG |
| SEQ ID NO: 3 | GEPRDG |
| SEQ ID NO: 4 | ENGEPRGDNY |
| SEQ ID NO: 5 | YESENGEPRGDNYRAY |
| SEQ ID NO: 6 | KRSR |
| SEQ ID NO: 7 | IKVV |
| SEQ ID NO: 8 | RGDC |
| SEQ ID NO: 9 | RGES |
| SEQ ID NO: 10 | Cyclo (RGDfV) |
| SEQ ID NO: 11 | RGDSP |
| SEQ ID NO: 12 | RGDSPC |
| SEQ ID NO: 13 | RGDS |
| SEQ ID NO: 14 | GRDC |
| SEQ ID NO: 15 | YRGDSPC |
| SEQ ID NO: 16 | (G)nRGD |
| SEQ ID NO: 17 | FHRRIKA |
| SEQ ID NO: 18 | RGDGGGKRSR |
| SEQ ID NO: 19 | KIPKASSVPTELSAISTLYL (20 AAs) |
| SEQ ID NO: 20 | GWQDWIIAPEGYAAYYCEGE |
| SEQ ID NO: 21 | KPCCAPTQLNAISVLYFDDS |
| SEQ ID NO: 22 | AISVLYFDDSSNVILKKYRN |

### REFERENCES

Balasundaram, G., Sato, M., and Webster, T. J., “Using hydroxyapatite nanoparticles and decreased crystallinity to promote osteoblast adhesion similar to functionalizing with RGD”, Biomaterials 27, 2798-2805 (2006)
Webster, T. J., and Ejiofor, J. U., “Increased osteoblast adhesion on nanophase metals: Ti, Ti6Al4V, and CoCrMo” Biomaterials 25, 4731-9 (2004)
Rezania, A., and Healy, K. E. “Integrin subunits responsible for adhesion of human osteoblast-like cells to biomimetic peptide surfaces”, J. Orthop. Res, 17: 615-623 (1999).
Ferris, D. M., Moodie, G. D., Dimond, P. M., Gioranni, C. W., Ehrlich, M. G., Valentini, R. F., “RGD-coated titanium implants stimulate increased bone formation in vivo”, Biomaterials 20:2323-2331 (1999).
Grimes, C. A., J. Phys. Chem. Letters B, 110, 16179-16184 (2006)
Villard, et al., J. Peptide Science, 12, 206-212 (2006)
Kasai, et al., Biopolymers, 76, 27-33 (2004)
Paulose, et al., J. Phys. Chem. B, 110(33), 16179-16184 (2006)
US Pub. No. US2007/0031388 (Geistlich)
U.S. Pat. No. 6,291,428
Bessho, K., Kusumoto, K., Fujimura, K., Konishi, Y., Ogawa, Y., Tani, Y., Iizuka, T. (1999). Comparison of recombinant and purified human bone morphogenetic protein. Br. J. Oral Maxillofac. Surg. 37, 2-5
Boden, S. D. (1999). Bioactive factors for bone tissue engineering. Clin. Orthop. Relat. Res. 367, 84-94.
Burt, D. W., Law, A. S. (1994). Evolution of the transforming growth factor-beta superfamily. Prog. Growth Factor Res. 5, 99-118.
Chen, P., Carrington, J. L., Hammonds, R. G., Reddi, A. H. (1991). Stimulation of chondrogenesis in limb bud mesoderm cells by recombinant human bone morphogenetic protein 2B (BMP-2B) and modulation by transforming growth factor beta 1 and beta 2. Exp. Cell Res. 195, 509-515.
Poynton, A. R., Lane, J. M. (2002). Safety profile for the clinical use of bone morphogenetic proteins in the spine. Spine. 27, 40-48.
Urist, M. R. (1965). Bone: formation by autoinduction. Science. 150, 893-899.
Wozney, J. M., Rosen, V., Byrne, M., Celeste, A. J., Moutsatsos, I., Wang, E. A. (1990). Growth factors influencing bone development. J. Cell Sci. 13, 149-156.
Bauer, S., Kleber, S, and Schmuki, P., “TiO₂ nanotubes: tailoring the geometry in H₃PO₄/HF electrolytes” Electrochemistry Communications 8, 1321-1325 (2006)
Oh, S., Daraio, C., Chen, L.-H., Pisanic, T. R., Finones, R. R., and Jin, S., “Significantly accelerated osteoblast cell growth on aligned TiO2 nanotubes”, J. Biomedical Materials Research, Part A, 6 Apr. 2006, 97-103.

1. A titanium or titanium alloy substrate coated with a molecular plasma deposited polypeptide wherein said substrate comprises a nanotubular structured surface.

2. The nanotubular surface of claim 1 wherein the molecular plasma is generated from a colloidal suspension or solution of the polypeptide generated at high voltage as a charged molecular corona.

3. The titanium alloy substrate of claim 1 wherein the titanium alloy is TiAlV, TiNi, TiAlNb, TiN, TiNbTaZr or Nitinol.

4. The nanotubular surface of claim 1 wherein the nanotubes are about 40 nm to about 500 nm in diameter.

5. The nanotubular surface of claim 4 wherein the nanotubes are about 70-130 nm in diameter.

6. The nanotubular surface of claim 1 wherein the nanotubes are about 50-500 nm in length.

7. The nanotubular surface of claim 1 wherein the deposited polypeptide is selected from RGDS (SEQ ID NO. 12); KRSR (SEQ ID NO. 6) and IKVAV (SEQ ID NO. 1) and combinations thereof.

8. The nanotubular surface of claim 7 wherein the deposited polypeptide is RGDS.

9. A method for promoting bone-forming cell adhesion to a substrate surface, comprising coating an anodized titanium surface with a molecular plasma deposited polypeptide selected from the group consisting of vitronectin, fibronectin, collagen, peptides SEQ ID NO:1, SEQ ID NO:2, SEQ ID NO: 3, BSP, BMP-2, and OP-1; and exposing the coated substrate surface to osteoblast cells wherein the osteoblast cells adhere to the surface and are capable of proliferating under in vitro or in vivo conditions.

10. The method of claim 9 wherein the polypeptide is RODS (SEQ ID No. 12); KRSR (SEQ NO. 6); IKVAV (SEQ ID NO. 1) or combinations thereof.

11. The method of claim 9 wherein the polypeptide is a protein comprising the amino acid sequence of SEQ ID NO. 12 or SEQ ID NO. 6.

12. The method of claim 9 wherein the anodized titanium surface comprises nanotubules having a diameter of about 40 up to about 120 nm.

13. A nanotubular titanium surface coated with a peptide comprising the amino acid sequence RGD to which osteoblast cells are attached.

14. A time-release bioactive material coated on a nanotubular titanium substrate wherein the bioactive material is non-covalently associated with the nanotubular landscape and releases with a t^{1/2} of about 3 days.
